(19) United States
(12) Patent Application Publication
(10) Pub. No.: US 2019/0125715 A1
(43) Pub. Date: May 2, 2019

(54) CDDO-Me AS A THERAPY FOR LUPUS

(71) Applicants: Chandra MOHAN, Sugarland, TX (US); Tianfu WU, Sugarland, TX (US); Michael ANDREEFF, Houston, TX (US); UNIVERSITY OF HOUSTON SYSTEM, Houston, TX (US)

(72) Inventors: Chandra Mohan, Sugarland, TX (US); Tianfu Wu, Sugarland, TX (US); Michael Andreeff, Houston, TX (US)

(21) Appl. No.: 15/600,064

(22) PCT Filed: Nov. 19, 2015
(86) PCT No.: PCT/US2015/061525

§ 371 Date: May 19, 2017

Related U.S. Application Data
(60) Provisional application No. 62/081,719, filed on Nov. 19, 2014.

(51) Int. Cl. A61K 31/277; A61P 37/06

(52) U.S. Cl. CPC A61K 31/277; A61P 37/06

(57) ABSTRACT

Triterpenoid drugs such as CDDO-Me can be used as to treat autoimmune diseases such as graft versus host disease, lupus, and lupus nephritis. Triterpenoid drugs can also be used to prevent the effects of an autoimmune disease.

### CROSS-REFERENCE TO RELATED APPLICATIONS

This application is the National Stage of International Application No. PCT/US2015/061525, filed Nov. 19, 2015, which claims priority to U.S. Provisional Application No. 62/081,719, filed Nov. 19, 2014, the entirety of which are hereby incorporated by reference.

### GOVERNMENT FUNDING

This invention was made with government support under grant AR-50812 awarded by The National Institutes of Health. The government has certain rights in the invention.

### FIELD

The present disclosure pertains to the treatment of autoimmune diseases. More specifically, the present disclosure is directed to ameliorating multiple phenotypes associated with lupus by blocking signaling pathways.

### BACKGROUND

Systemic lupus erythematosus (SLE) is a highly complex autoimmune disease characterized by hyperproliferation and hyperactivation of lymphocytes, autoantibody production, and eventually end-organ damage. Several lines of evidence have indicated that specific signaling pathways are involved in the pathogenesis of lupus. For example, overexpression of both phosphatidylinositol 3-kinase (PI3K) (1) and the antiapoptotic molecule Bcl-2 (2), as well as haplo-insufficiency of the tumor suppressor PTEN (Pten^{+/-}) (3) have been shown to cause lymphoproliferative lupus. Our previous studies demonstrated that multiple signaling pathways are upregulated in lupus B cells, including the AKT pathway, MAPK pathway, JAK/STAT pathway, cyclin-dependent kinase pathway, NF-κB pathway, and pathways downstream of some antiapoptotic Bcl-2 family members (4). Given that lupus is associated with the activation of multiple signaling axes, therapies targeting multiple activated signaling cascades can prove to be more effective in curtailing this disease.

In lupus, B cells, T cells, and even myeloid cells are hyperproliferative and hyperactive. These hyperactivated immune cells can infiltrate organs, causing tissue damage, resulting in end-organ problems such as nephritis. Previous studies have demonstrated that despite the distinct genetic backgrounds of mouse models used for studying spontaneous lupus, these different strains share the up-regulation of similar cell signaling pathways involving PI3K/Akt/mammalian target of rapamycin (mTOR), MAP kinases, STAT-3/STAT-5, NF-κB, multiple Bcl-2 family members, and various cell cycle molecules in B cells (4). Several key signaling molecules, including NF-κB (20), STAT-3 (21), Ca²⁺/calmodulindependent protein kinase IV (22), and Syk (23), have also been observed to be altered in lupus T cells. Furthermore, several of these signaling intermediates are positive regulators of a number of inflammatory cytokines and chemokines. Hence, intervention in leukocyte signaling pathways might be beneficial in the treatment of lupus.

Triterpenoids are natural plant products generated by the cyclization of squalene and are used for medicinal purposes in many Asian countries, since they have been reported to have anticarcinogenic activity (24-27). Because the biologic activities of some of the natural triterpenoids are not strong enough, new analogs of these molecules have been chemically synthesized in an attempt to produce more potent agents. A novel synthetic triterpenoid derived from oleanolic acid, 2-cyano-3,12-dioxoolean-1,9-dien-28-oic acid (CDDO), has been shown to be a more potent antitumor and antiinflammatory agent than its natural plant-derived analogs (5,6). CDDO was found to inhibit the proliferation of many human cancer cells and to suppress the ability of various inflammatory cytokines such as interferon-γ (IFNγ), interleukin-1 (IL-1), and tumor necrosis factor α (TNFα).

CDDO-Me is a methyl derivative of CDDO that was found to be as active as CDDO in suppressing the increased production of nitric oxide by IFNγ in mouse macrophages (28). CDDO-Me, the C-28 methyl ester of CDDO, also known as bardoxolone methyl, has been shown to inactivate STAT-3 signaling (7,8), inhibit mitochondrial electron transport via perturbations in inner mitochondrial membrane integrity (9), block the NF-κB pathway (10), induce apoptosis by disrupting intracellular redox balance (11), induce the proapoptotic Bax protein (12), inhibit the activation of ERK-1/2 (13), and block Bcl-2 phosphorylation (12). Additionally, CDDO-Me protects against lipopolysaccharide (LPS)-induced inflammatory responses via activation of the NF-E2-related factor 2 (Nrf2)-dependent antioxidative pathway (14). Very recently, CDDO-Me has been shown to effectively sustain increases in the estimated glomerular filtration rate in patients with advanced chronic kidney disease and type 2 diabetes mellitus in a phase II clinical trial (15).

Furthermore, there are a number of studies showing that CDDO-Me can block selected signaling pathways. CDDO-Me has been identified as a potent caspase mediated apoptosis inducer in human lung cancer in acute myelogenous leukemia (12,29). CDDO-Me has also been shown to directly inhibit both JAK-1 and STAT-3 (7) and to inhibit the NF-κB pathway through direct inhibition of IKKβ on Cys179 (10). This compound has also been shown to inhibit IB kinase and to enhance apoptosis induced by TNF and chemotherapeutic agents through down-regulation of NF-κB-regulated gene products in human leukemia cells (30).

Current treatment options for lupus are far from optimal. However, it has been elucidated that phosphatidylinositol 3-kinase/Akt/mammalian target of rapamycin, MEK-1/ERK-1,2, p38, STAT-3, STAT-5, NF-κB, multiple Bcl-2 family members, and various cell cycle molecules are overexpressed in splenic B cells in an age-dependent and gene dose-dependent manner in mouse strains with spontaneous lupus. Since the synthetic triterpenoid methyl-2-cyano-3,12-dioxooleana-1,9-dien-28-oate (CDDO-Me) has been shown to inhibit AKT, MEK-1/2, and NF-κB, and to induce caspase mediated apoptosis, it would be advantageous to determine if triterpenoids are effective in treating lupus for clinical applications.

### SUMMARY

An embodiment of the disclosure is a method of treatment of a subject with an autoimmune disease comprising administering CDDO-Me to a subject, wherein the treatment results in suppression of activation of lymphocytes. In an embodiment, the lymphocytes are CD4+ T cells. In an embodiment, the lymphocytes are B cells. In an embodiment, administering CDDO-Me blocks signaling pathways in lymphocytes. In an embodiment, administering CDDO-Me inhibits at least one selected from the group consisting of AKT, MEK-1/2, NF-κB and a combination thereof in lymphocytes. In an embodiment, administering CDDO-Me induces apoptosis of the lymphocytes. In an embodiment, administering CDDO-Me reduces autoantibody production. In an embodiment, administering CDDO-Me reverses lupus nephritis by activation of a NRF2-mediated antioxidative. In an embodiment, the autoimmune disease is lupus. In an embodiment, the autoimmune disease is graft versus host disease. In an embodiment, the autoimmune disease is lupus nephritis

An embodiment of the disclosure is a method of prevention of an autoimmune disease in a subject using CDDO-Me, comprising administering CDDO-Me to a subject, wherein the CDDO-Me suppresses activation of lymphocytes. In an embodiment, the lymphocytes are CD4+ T cells. In an embodiment, the lymphocytes are B cells. In an embodiment, administering CDDO-Me inhibits in lymphocytes at least one selected from the group consisting of AKT, MEK-1/2, NF-κB and a combination thereof. In an embodiment, the CDDO-Me induces apoptosis of the lymphocytes. In an embodiment, the CDDO-Me reduces autoantibody production. In an embodiment, the autoimmune disease is lupus. In an embodiment, the autoimmune disease is graft versus host. In an embodiment, the autoimmune disease is lupus nephritis.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

In order that the manner in which the above-recited and other enhancements and objects of the invention are obtained, we briefly describe a more particular description of the invention briefly rendered by reference to specific embodiments thereof which are illustrated in the appended drawings. Understanding that these drawings depict only typical embodiments of the invention and are therefore not to be considered limiting of its scope, we herein describe the invention with additional specificity and detail through the use of the accompanying drawings in which:

FIG. 1. Attenuation of disease in B6.Sle1.Sle3 mice with spontaneous lupus treated with methyl-2-cyano-3,12-dioxooleana-1,9-dien-28-oate (CDDO-Me). Two-month-old female B6.Sle1.Sle3 mice (n=20 per group) were treated with CDDO-Me or placebo (sesame oil) as indicated. FIG. 1A and FIG. 1B, Amelioration of splenomegaly, as indicated by spleen weight (Fig. A) and splenic cell number (FIG. 1B) in mice 60 days after treatment with CDDO-Me. In FIG. 1A, circles represent individual mice; horizontal lines show the mean. In FIG. 1B, bars show the mean±SEM. FIG. 1C, Suppression of the expansion of activated CD4+ T cells in B6.Sle1.Sle3 mice examined 60 days after treatment. Flow cytometry plots show results from a representative experiment. FIG. 1D, Hematoxylin and eosin staining of kidney sections from a mouse treated with CDDO-Me and a mouse treated with placebo. Results are representative of several similar experiments (n=10). Reduction in proteinuria (FIG. 1E), glomerulonephritis (GN) score (FIG. 1F), and blood urea nitrogen (BUN) (FIG. 1G) in B6.Sle1.Sle3 mice after 2 months of treatment with CDDO-Me. FIG. 1H-Fig. K, Attenuation of serum IgG autoantibody levels in B6.Sle1.Sle3 mice treated with CDDO-Me. In FIG. 1E-FIG. 1K, data are shown as box plots. Each box represents the 25th to 75th percentiles. Lines inside the boxes represent the median. Lines outside the boxes represent the 10th and 90th percentiles. Data are shown for mice at the age of 2 months (day 0) and 4 months (day 60). Serially diluted sera from B6.Sle1.lpr mice were used for plotting a standard curve, and the highest standard was set at 100 AU. *=P<0.05; **=P<0.01; ***=P<0.001, by Student's t-test Anti-dsDNA=anti-double-stranded DNA; anti-ssDNA=anti-single-stranded DNA; anti-glom=antiglomerular antibodies; AU=arbitrary units.

FIG. 2. Inhibition of selected signaling axes in splenic B cells (FIG. 2A) and T cells (FIG. 2B) from female B6.Sle1.Sle3 mice treated with methyl-2-cyano-3,12-dioxooleana-1,9-dien-28-oate (CDDO-Me). At the end of the 60-day CDDO-Me treatment period (i.e., when all mice were 4 months of age), splenic B cells or T cells were purified using magnetic beads, and cell lysates were examined for signaling status by Western blot analysis. Western blot results were quantified using ImageJ software and plotted as bar graphs. Bars show the mean±SEM band intensity (n=10 mice per group). *=P<0.05; **=P<0.01; ***=P<0.001, by Student's t-test.

FIG. 3. Up-regulation of the NF-E2-related factor 2 (Nrf2) signaling pathway in the kidneys of B6.Sle1.Sle3 mice after methyl-2-cyano-3,12-dioxooleana-1,9-dien-28-oate (CDDO-Me) treatment. Kidneys were collected from both CDDO-Me-treated and placebo-treated female B6.Sle1.Sle3 mice, and kidney lysates were used for Western blot analysis. Western blot results were quantified using ImageJ software and plotted as bar graphs. Bars show the mean±SEM band intensity (n=4-5 mice per group). ***=P<0.001 by Student's t-test. NQO-1=NAD(P)H quinone oxidoreductase 1. GCLC=glutamate cysteine ligase catalytic subunit; GCLM=glutamate cystein ligase modifier subunit.

FIG. 4. Amelioration of disease by methyl-2-cyano-3,12-dioxooleana-1,9-dien-28-oate (CDDO-Me) in the MRL/lpr mouse model of lupus. (FIG. 4A), Significant reduction in splenomegaly in mice treated with CDDO-Me for 2 months compared to mice treated with water or sesame oil. Significantly reduced spleen weight was also noted in the mice treated with dexamethasone (DEX). FIG. 4B and FIG. 4C, Significant decrease in anti-double-stranded DNA (anti-dsDNA) antibody levels (FIG. 4B) and proteinuria and glomerulonephritis (GN) score (FIG. 4C) in mice treated with CDDO-Me compared to mice treated with water or sesame oil. In FIG. 4A-FIG. 4C, data are shown as box plots. Each box represents the 25th to 75th percentiles. Lines inside the boxes represent the median. Lines outside the boxes represent the 10th and 90th percentiles. FIG. 4D, Reduced phosphorylation of MEK and ERK-1/2 and increased phosphorylation of NF-E2-related factor 2 (Nrf2) in both splenic B cells and T cells from mice treated with CDDO-Me. *=P<0.05.

FIG. 5A-FIG. 5C, Significant reduction in cellular reactive oxygen species (ROS) in both splenic CD4+ T cells (FIG. 5B) and CD11b+ cells (FIG. 5C) but not B220+ B cells (FIG. 5A) isolated from 4-month-old MRL/lpr mice treated with methyl-2-cyano-3,12-dioxooleana-1,9-dien-28-oate (CDDO-Me). FIG. 5D-FIG. 5F, Positive correlation of the reduction in ROS with the decreased phosphorylation of ERK-1/2 (FIG. 5D) and MEK (FIG. 5E), and negative correlation of the reduction in ROS with the increase in NF-E2-related factor 2 (Nrf2) (FIG. 5F). DEX=dexamethasone; AU=arbitrary units.

### DETAILED DESCRIPTION

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The synthetic triterpenoid CDDO-Me or a placebo was administered to 2-month-old B6.Sle1.Sle3 mice or MRL/lpr mice, which develop spontaneous lupus. All mice were phenotyped for disease. The CDDO-Me-treated mice exhibited significantly reduced splenic cellularity, with decreased numbers of both CD4+ T cells and activated CD69+/CD4+ T cells compared to the placebo-treated mice. These mice also exhibited a significant reduction in serum autoantibody levels, including anti-double-stranded DNA (anti-dsDNA) and antiglomerular antibodies. Finally, CDDO-Me treatment attenuated renal disease in mice, as indicated by reduced 24-hour proteinuria, blood urea nitrogen, and glomerulonephritis. At the mechanistic level, CDDO-Me treatment dampened MEK-1/2, ERK, and STAT-3 signaling within lymphocytes and oxidative stress. Importantly, the NF-E2-related factor 2 pathway was activated after CDDO-Me treatment, indicating that CDDO-Me can modulate renal damage in lupus via the inhibition of oxidative stress. These findings underscore the importance of AKT/MEK-1/2/NF-κB signaling in engendering murine lupus. The blockade of multiple signaling nodes and oxidative stress can effectively prevent and reverse the hematologic, autoimmune, and pathologic manifestations of lupus.

CDDO-Me has a reproducible impact on suppressing murine lupus and lupus nephritis when administered before disease develops, and more importantly, after the onset of disease. This agent appears to be operating by suppressing multiple cell signaling axes in leukocytes (and possibly other tissues) and countering oxidative stress. Given the efficacy of this agent in modulating immune cell signaling as well as lupus nephritis, this can be an attractive option to pursue in the context of human lupus therapies.

In an embodiment, triterpenoids such as CDDO-Me are used to treat a subject with an autoimmune disease, wherein the treatment results in the suppression of activation of lymphocytes. The lymphocytes can include, but are not limited to, CD4+ T cells or B cells. In an embodiment, the lymphocyte population undergoes apoptosis, does not become activated, or autoantibodies are not generated. The triterpenoid often inhibits AKT, MEK-1/2, NF-κB, or a combination thereof. CDDO-Me can target multiple signaling pathways in multiple cell types, including but not limited to T cells, B cells, lung cells, breast cells, myeloid leukemia cells, and colorectal cancer cells. Therefore, it was examined whether it had the potential to suppress lymphoproliferation, autoantibody production, and renal inflammation in murine lupus.

In an embodiment, triterpenoids, such as CDDO-Me, can be involved in suppressing the increased production of nitric oxide by IFNγ in mouse macrophages, inactivating STAT-3 signaling (7,8); inhibiting mitochondrial electron transport via perturbations in inner mitochondrial membrane integrity (9); blocking the NF-κB pathway (10); inducing apoptosis by disrupting intracellular redox balance (11); inducing the proapoptotic Bax protein (12); inhibiting the activation of ERK-1/2 (13); and blocking Bcl-2 phosphorylation (12); protecting against lipopolysaccharide (LPS)-induced inflammatory responses via activation of the NF-E2-related factor 2 (Nrf2)-dependent antioxidative pathway (14); sustaining increases in the estimated glomerular filtration rate in patients with advanced chronic kidney disease and type 2 diabetes mellitus (15); inhibiting AKT, MEK-1/2, and NF-κB; inducing caspase mediated apoptosis; inhibiting both JAK-1 and STAT-3 (7); inhibiting the NF-κB pathway through direct inhibition of IKKβ on Cys179 (10); inhibiting IB kinase; and enhancing apoptosis induced by TNF and chemotherapeutic agents through down-regulation of NF-κB-regulated gene products in human leukemia cells.

In an embodiment, triterpenoids can be used to treat autoimmune diseases. In an embodiment, the triterpenoids include, but are not limited to, CDDO, CDDO-M, CDDO-Im, ursolic acid, oleanolic acid, betulinic acid, celastrol, pristimerin, lupeol, avicins, squalene, and lanosterol.

In an embodiment, triterpenoids can be used to prevent autoimmune diseases. In an embodiment, the triterpenoids include, but are not limited to, CDDO, CDDO-M, CDDO-Im, CDDO-dhTFEA, ursolic acid, oleanolic acid, betulinic acid, celastrol, pristimerin, lupeol, avicins, squalene, and lanosterol.

In an embodiment, triterpenoids, including but not limited to CDDO-Me, could be useful in treatment of graft versus host disease, lupus, lupus nephritis, kidney disease, diabetes mellitus, cancer, and kidney damage. In an embodiment, triterpenoids could be used to treat any autoimmune disease including but not limited to rheumatoid arthritis, systemic lupus erythematosus, Hashimoto's disease, Addison's disease, Grave's disease, celiac sprue disease, vitiligo, scleroderma, psoriasis, inflammatory bowel diseases, pernicious anemia, reactive arthritis, Sjogren's syndrome, graft versus host disease, lupus, lupus nephritis, kidney disease, diabetes mellitus, kidney damage, and type 1 diabetes.

In an embodiment, triterpenoids, including but not limited to CDDO-Me, could be useful in prevention of graft versus host disease, lupus, lupus nephritis, kidney disease, diabetes mellitus, cancer, and kidney damage. In an embodiment, triterpenoids could be used to prevent any autoimmune disease including but not limited to rheumatoid arthritis, systemic lupus erythematosus, Hashimoto's disease, Addison's disease, Grave's disease, celiac sprue disease, vitiligo, scleroderma, psoriasis, inflammatory bowel diseases, pernicious anemia, reactive arthritis, Sjogren's syndrome, graft versus host disease, lupus, lupus nephritis, kidney disease, diabetes mellitus, kidney damage, and type 1 diabetes.

### EXAMPLES

### Example 1. Methods

Animals: C57BL/6 (B6), MRL/lpr, and NZM2410 were used in this study. The derivation of B6-congenic mice bearing different NZM2410-derived lupus susceptibility intervals has been described previously (16). B6.Sle1.Sle3 mice, which are bicongenic for the 2 lupus susceptibility intervals Sle1 and Sle3, were previously derived by intercrossing the respective monocongenic strains (4). Both male and female mice were used, and any observed sex differences are noted.

Flow cytometric analysis and antibodies: Splenocytes were depleted of red blood cells using a lysis buffer (containing 0.15M NH4Cl, 10 mM KHCO3, and 0.1 mM Na₂EDTA, pH 7.2), and single-cell suspensions were prepared for flow cytometric analysis. Flow cytometric analysis was performed as described previously (17).

Purification of splenic B220+B cells and CD4+T cells: Spleens were harvested from mice postmortem, and single-cell suspensions were prepared by crushing spleens between frosted glass slides. Red blood cells were lysed using ACK lysis buffer (Invitrogen) followed by 2 washes in phosphate buffered saline plus 0.5% bovine serum albumin. B220+B cells were purified by positive selection from this total splenocyte suspension with B220 microbeads (Miltenyi Biotec). CD4+T cells were purified by positive selection from total splenocytes with CD4 microbeads according to the recommendations of the manufacturer (Miltenyi Biotec).

Enzyme-linked immunosorbent assay (ELISA) for autoantibodies: The anti-double-stranded DNA (anti-dsDNA), antihistone, and antihistone/DNA ELISAs were carried out as previously described (17). Raw optical density was converted to units/milliliter, using a positive control serum derived from a B6.Sle1.lpr mouse, and arbitrarily setting the reactivity of a 1:100 dilution of this serum to 100 units/ml. Test sera with reactivity stronger than the standard were diluted further and re-assayed. The glomerular-binding ELISA was performed as described previously (17), using sonicated rat glomeruli as the substrate.

Western blot analysis: Purified B cells or T cells were lysed using 20 mM Tris HCl (pH 7.5), 150 mM NaCl, 1 mM Na₂EDTA, 1 μg/ml leupeptin, 1% Triton X-100, 1 mM phenylmethylsulfonyl fluoride, and 1 mM Na₃VO₄. Total protein was quantified by the Bradford method, and 10 μg was loaded per lane onto sodium dodecyl sulfate-polyacrylamide gel electrophoresis gels. The following primary antibodies were used: p-STAT3^{S727} (catalog no. 9134), p-ERK-1/2^{T202/Y204} (catalog no. 4376), ERK-1/2 (catalog no. 4695), p-NF-κB^{S536} (catalog no. 3031), p-MEK-1/2S217/221 (catalog no. 9121), MEK-1/2 (catalog no. 9122), AKT (catalog no. 9272), p-AKT^{T308} (catalog no. 9275), α-tubulin (catalog no. 2144) (all from Cell Signaling Technology), β-actin (catalog no. RGM2; Advanced ImmunoChemical), and Bcl-2 (catalog no. sc-23960; Santa Cruz Biotechnology). Antibodies to Nrf2 (catalog no. ab71890), NAD(P)H quinone oxidoreductase 1 (NQO-1; catalog no. ab2346), GCLC (catalog no. ab53179), and GCLM (catalog no. ab81445) were purchased from Abcam. Horseradish peroxidase-conjugated secondary antibodies and an ECL Plus detection kit (Amersham) were used to develop the blot. For Western blot analysis of purified B cells and T cells, cells were pooled from 3-4 mice for each lane. The respective band intensities were measured using ImageJ software version 1.37 (National Institutes of Health; online at http://rsb.info.nih.gov/ij), and normalized against the corresponding β-actin or α-tubulin levels. Where samples from different strains were compared, normalized band intensities were expressed as ratios, relative to the corresponding levels in B6 mice.

Statistical analysis: Statistical comparisons were performed using Student's paired or unpaired t-tests, as appropriate, using SigmaStat (Jandel Scientific). For all experiments, results are shown as the mean±SEM. P values less than 0.05 were considered significant.

In vivo experiments: In the first preventive experiment, CDDO-Me was diluted in sesame oil. Two-month-old B6.Sle1.Sle3 mice (n=20) received CDDO-Me at a final dose of 3 mg/kg or vehicle alone (i.e., placebo), by oral gavage 3 times per week for a period of 2 months. In a confirmatory preventive study, 2-month-old MRL/lpr mice (n=8 per group) were orally administered CDDO-Me (3 mg/kg), sesame oil, water, or dexamethasone (1 mg/kg) 3 times a week for 2 months. Serum and 24-hour urine samples were obtained on days 0, 14, and 60. All serum samples were subjected to ELISA for autoantibodies, and urine samples were assayed for total protein as previously described (17,18). On day 60, when mice were killed, the cellular composition of the spleen and lymph nodes was determined by flow cytometry, and the kidneys were examined for pathology, as described below. In addition, the expression of various signaling molecules in the spleens and kidneys of the treated mice was assayed by Western blotting, as described above. In the treatment experiment, 7-month-old NZM2410 mice with proteinuria (n=4-7 per group) were treated with CDDO-Me (3 mg/kg) or placebo by oral gavage 5 times per week for a period of 2 months. Proteinuria, autoantibody production, spleen weight, and survival rates were assessed.

Cellular reactive oxygen species (ROS) detection: Cellular ROS detection was performed using a flow cytometry-based method with a DCFDA-Cellular Reactive Oxygen Species Detection Assay Kit (catalog no. ab113851; Abcam). For the in vivo experiments, splenocytes were harvested from MRL/lpr mice after treatment with CDDO-Me, placebos, or dexamethasone. An aliquot of 1.5×10⁵ cells was stained with 20 μM DCFDA or antibodies to cell surface markers (B220, CD4, and CD11b). DCF was excited with a 488-nm laser and detected at 535 nm. For the in vitro experiments, splenocytes were isolated from 4-month-old MRL/lpr mice and treated with a STAT-3 inhibitor (1 μM cucurbitacin I; Santa Cruz Biotechnology) or MEK-1 inhibitor (50 μM PD98059; Cell Signaling Technology) for 4 hours. These cells were then stained with 20 μM DCFDA or antibodies to cell surface markers (B220, CD4, and CD11b) for flow cytometric analysis.

Renal disease: Twenty-four-hour urine samples were collected using metabolic cages. The total amount of urinary protein was assayed using a Coomassie-based assay (Pierce). When mice were killed, kidneys were fixed, sectioned, and stained with hematoxylin and eosin and periodic acid-Schiff. At least 100 glomeruli were examined per section by light microscopy for evidence of inflammation and/or tissue damage, and graded as previously described (18), in a blinded manner. The occurrence of any mesangiopathic, capillary hyaline, proliferative, membranous, or crescentic glomerular changes was also noted.

### Example 2. Suppression of the Hyperactivation of B Cells and CD4+ T Cells by CDDO-Me

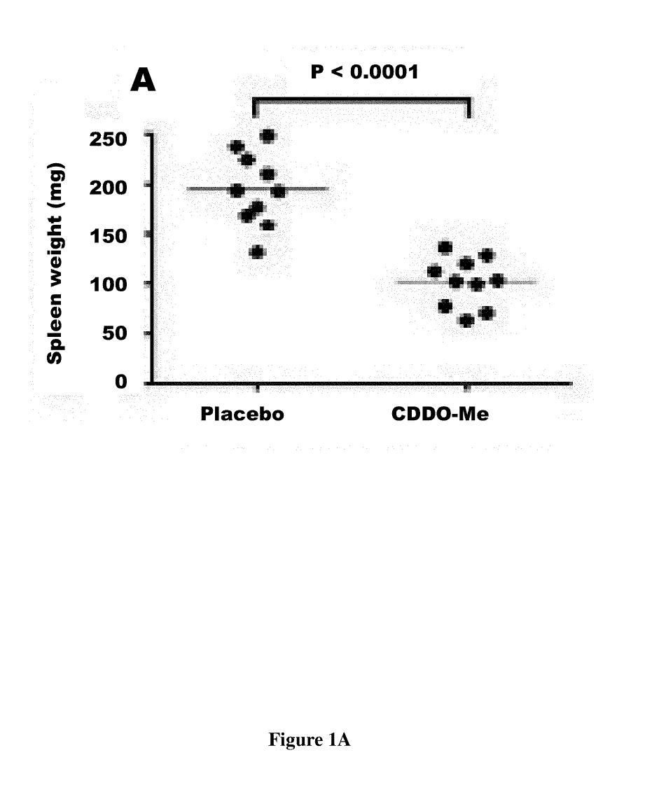

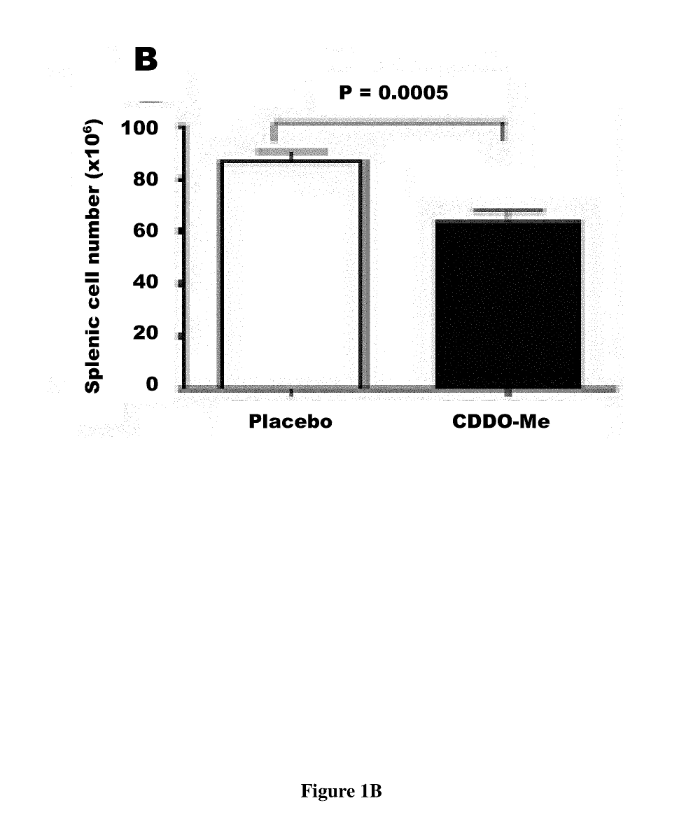

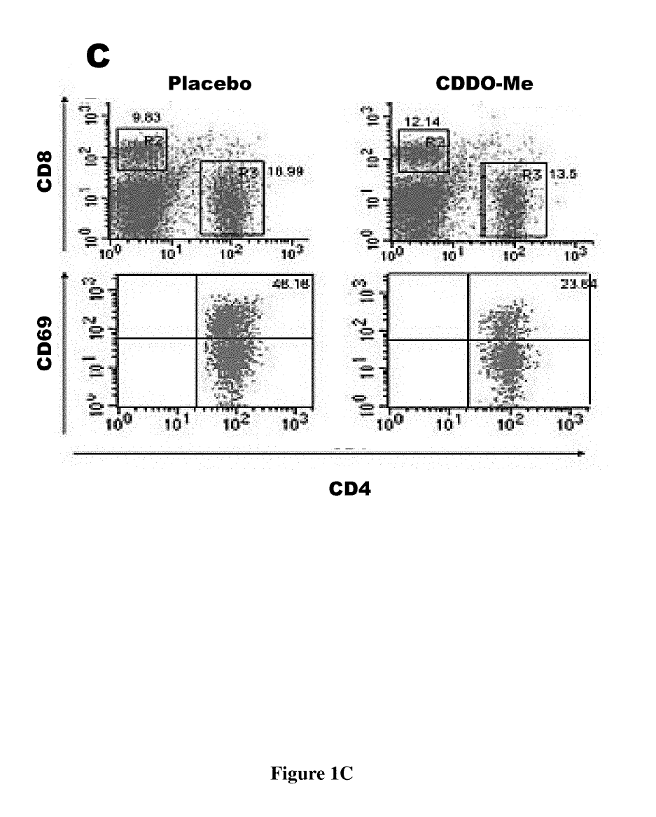

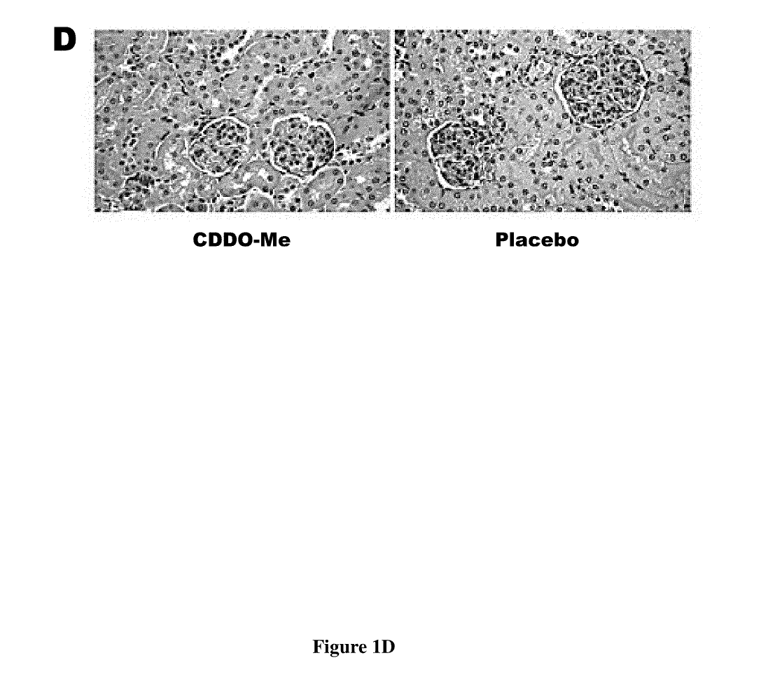

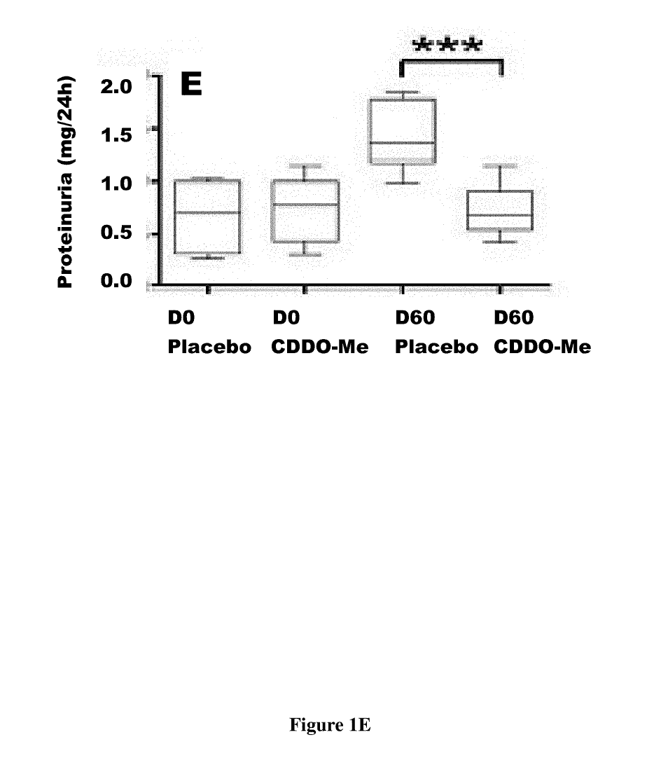

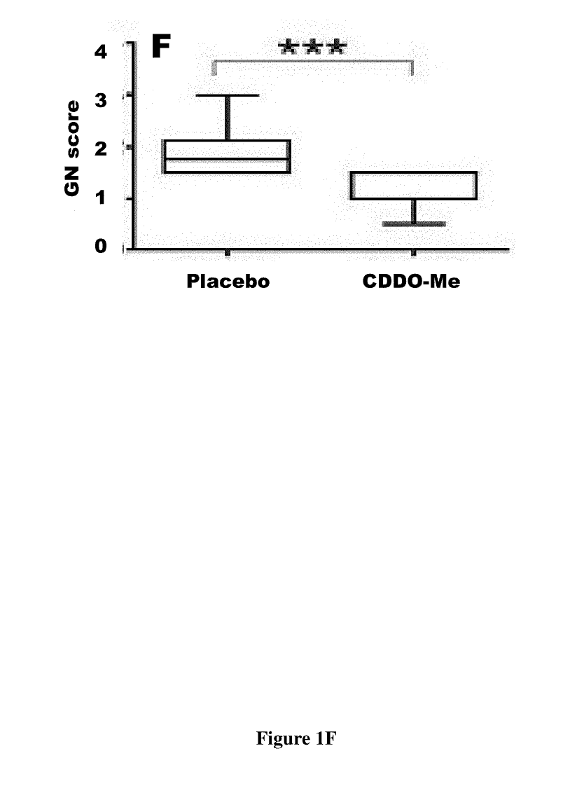

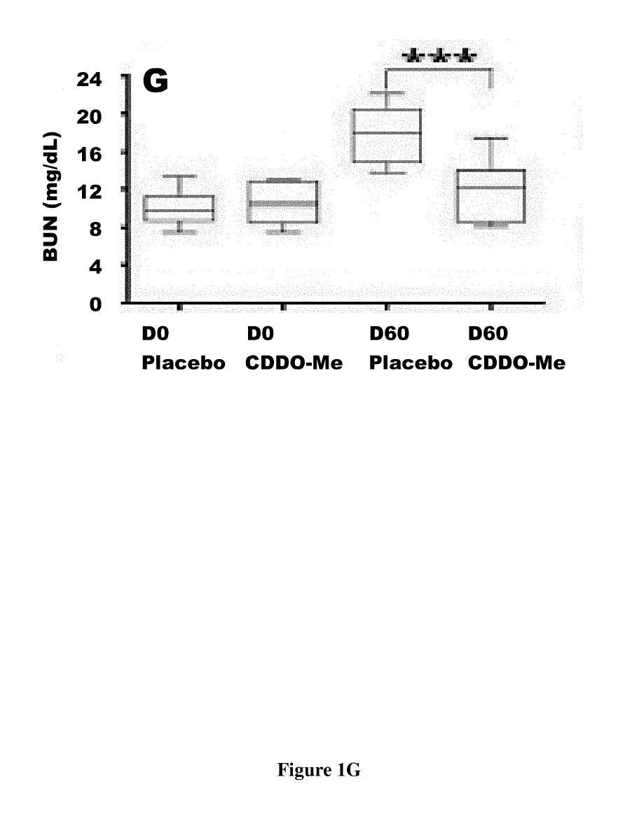

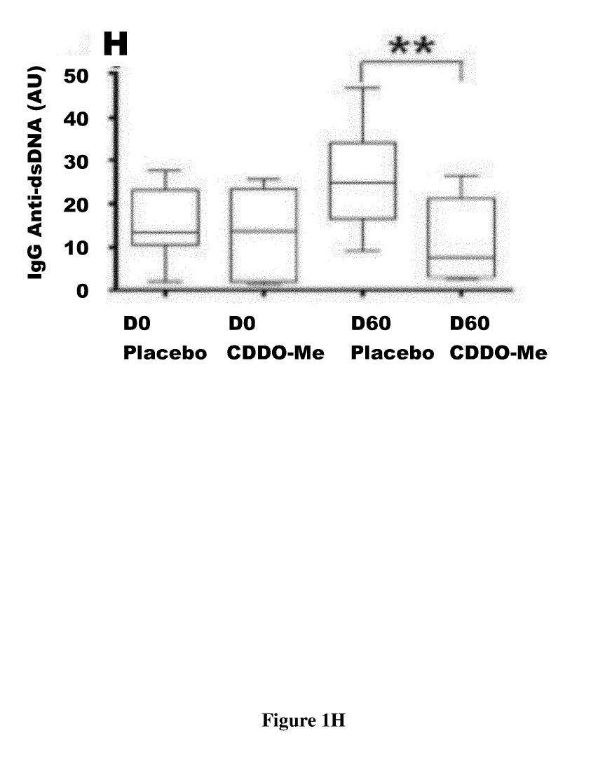

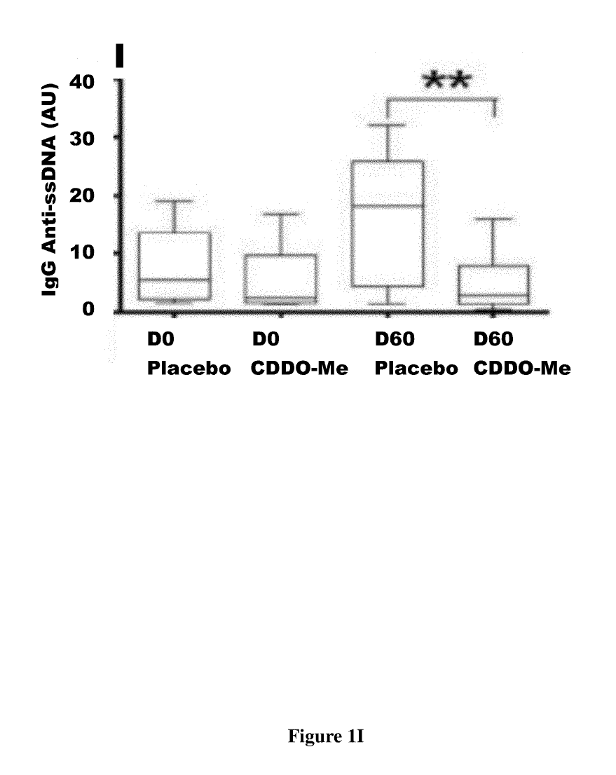

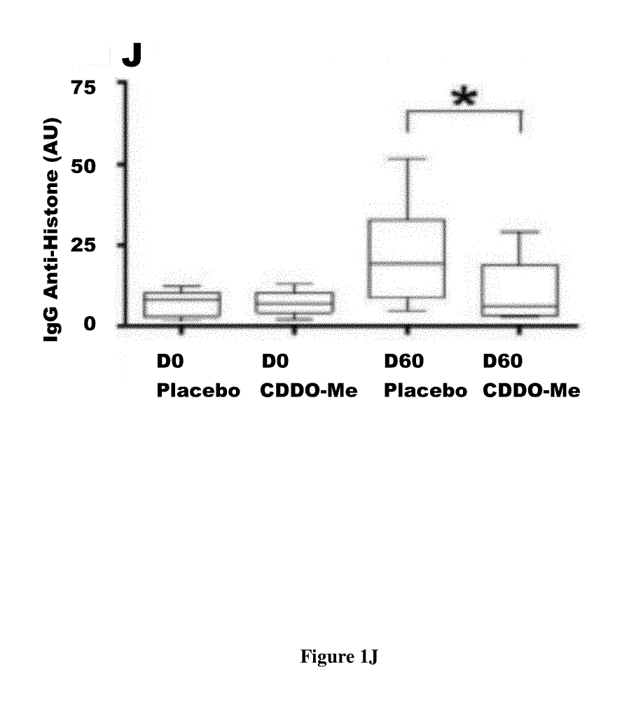

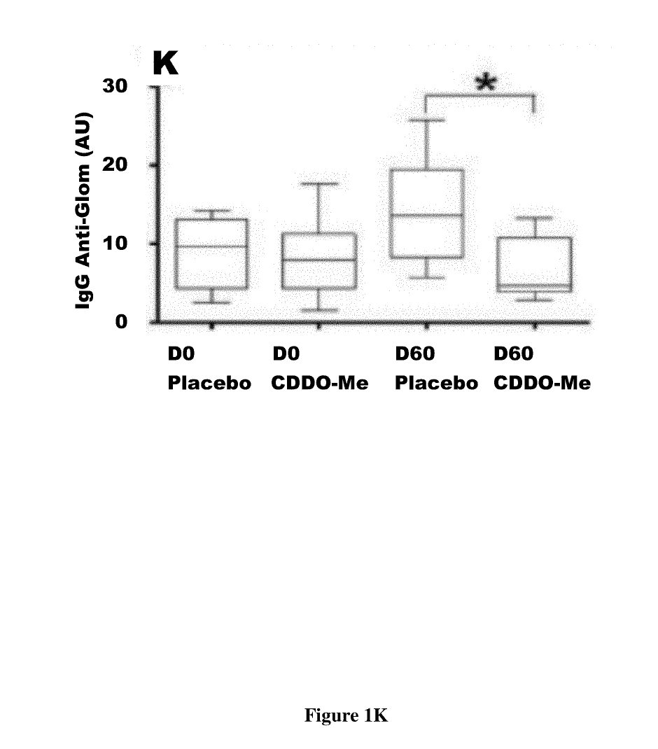

Given the previous demonstration that CDDO-Me suppresses cell proliferation, it was investigated whether CDDO-Me suppressed the development of splenomegaly in mice with lupus. To address this, 2-month-old female B6.Sle1.Sle3 mice were treated for 60 days with CDDO-Me or placebo, and then spleen size and cellularity were assessed in both groups. Notably, the overall spleen weights in the CDDO-Me-treated group were decreased almost 50% compared to the placebo-treated group (FIG. 1A). Consistent with these findings, the total number of splenocytes was also decreased in the CDDO-Me-treated group compared to the placebo-treated group (FIG. 1B).

Next, it was investigated which cell populations were significantly suppressed by CDDO-Me. As expected, among splenic T cells, the percentage of CD4+ T cells was decreased (mean±SEM 12.1±0.35% versus 15.1±1.2%; P=0.021), while the percentage of CD8+ T cells was increased (9.73±0.4% versus 6.8±1.1%; P=0.023) in the CDDO-Me-treated group (FIG. 1C). The absolute number of total splenic CD4+ T cells was also decreased (18.7±3.8 million versus 39.0±2.0 million; P<0.0001) in the CDDO-Me-treated group (Table 1). Within the CD4+ T cell compartment, the activated population (CD69+) was significantly deceased in the CDDO-Me-treated group compared to the control group (FIG. 1C and Table 1). Of note, besides the dramatic reduction in and deactivation of CD4+ T cells, the absolute cell numbers (if not percentages) of splenic B220+ B cells (both mature and immature B cells, and B1a cells) were also decreased with CDDO-Me treatment (Table 1). B cell activation, as gauged by surface CD86 expression, was also markedly reduced following CDDO-Me treatment (6.48±0.42 versus 8.72±0.45 mean fluorescence intensity units; P<0.002). Importantly, after CDDO-Me treatment, the cell numbers and activation status of splenic B cells and T cells and their subsets were reversed to normal, similar to the phenotypes previously seen in healthy B6 mice (17).

TABLE 1
Activation status and lymphocyte subsets in B6.Sle1.Sle3mice treated
with CDDO-Me or placebo*

| | Placebo | CDDO-Me | P |
|---|---|---|---|
| Total spleen cell count (10⁶) | 146.8 ± 2.5 | 89.1 ± 2.3 | 0.0005 |

Splenic T cells

| | | | |
|---|---|---|---|
| CD4 T cells | 39.0 ± 2.0 | 18.7 ± 3.8 | <0.0001 |
| CD8 T cells | 9.9 ± 1.3 | 8.6 ± 2.4 | NS |
| CD69+/CD4 T cells | 14.5 ± 0.9 | 5.1 ± 0.6 | <0.0001 |
| CD69+/CD8 T cells | 1.4 ± 0.3 | 0.6 ± 0.1 | 0.008 |

Splenic B cells

| | | | |
|---|---|---|---|
| B220+, AA4.1+ (mature) | 64.8 ± 4.5 | 45.3 ± 3.2 | 0.013 |
| Follicular B cells | 40.8 ± 2.7 | 30.9 ± 1.6 | 0.017 |
| Marginal zone B cells | 8.3 ± 0.5 | 4.0 ± 0.2 | 0.062 |
| B1a cells | 7.5 ± 0.9 | 4.0 ± 0.3 | 0.013 |
| B220+, AA4.1+ | 14.0 ± 1.3 | 10.4 ± 1.1 | 0.032 |
| (immature) | | | |
| T1 immature | 2.0 ± 0.2 | 1.0 ± 0.1 | 0.02 |
| T2 immature | 4.5 ± 0.3 | 3.1 ± 0.2 | NS |
| T3 immature | 4.2 ± 0.2 | 4.0 ± 0.2 | 0.061 |

*Values are the mean SEM (n = 10 mice per group). All data were obtained using 4-month-old mice. CDDO-Me = methyl-2-cyano-3,12-dioxooleana-1,9-dien-28-oate; NS = not significant.

### Example 3. Amelioration of Kidney Disease, as Manifested by Reduced Proteinuria, Blood Urea Nitrogen (BUN), and Glomerulonephritis (GN), in B6.Sle1.Sle3 Mice Treated with CDDO-Me

It was examined whether the administration of CDDO-Me reduces renal damage in murine lupus nephritis. On day 60 after placebo or CDDO-Me treatment, urine was collected and tested for proteinuria. Compared to the placebo-treated group, the CDDO-Me-treated mice showed significantly reduced proteinuria (FIG. 1E). Examination of the mouse kidneys clearly demonstrated that CDDO-Me treatment resulted in lower GN scores than placebo treatment. Microscopic analysis revealed increased cellularity in the glomeruli of mice treated with placebo compared to those treated with CDDO-Me, indicating the presence of more inflammation and greater numbers of infiltrating cells in the placebo-treated mice. CDDO-Me treatment also led to reduced BUN levels, further indicating that renal function was improved in these mice (FIG. 1F and FIG. 1G). Most importantly, all parameters of renal disease were reversed to normal, similar to the phenotypes previously seen in healthy B6 mice (17).

CDDO-Me is beneficial in suppressing hyperactivation of immune cells, particularly CD4+ T cells (FIG. 1 and Table 1). In a murine acute graft-versus-host disease model, CDDO-Me exhibited an increased ability to inhibit allogeneic T cell responses and induce cell death of alloreactive T cells in vitro (31). In a transgenic adenocarcinoma of the mouse prostate (TRAMP) cancer model, CDDO-Me induced apoptosis in TRAMP C1 cells, as revealed by the increased expression of annexin V and cleavage of procaspases 3, 8, and 9; CDDO-Me also inhibited NF-κB-regulated antiapoptotic Bcl-2, BclxL, and X-linked inhibitor of apoptosis protein (32,33). Additionally, CDDO-Me participates in the induction of apoptosis in acute myeloid leukemia (34). Both splenic B cells and T cells were decreased in the CDDO-Me-treated mice compared to the placebo treated controls, suggesting that CDDO-Me might induce apoptosis in splenic B cells and T cells, thereby subduing autoimmunity.

### Example 4. Amelioration of Autoantibody Production in B6.Sle1.Sle3 Mice Treated with CDDO-Me

The reduced activation of lymphocytes was associated with a reduction in the production of autoantibodies such as anti-dsDNA, anti-ssDNA, antihistone, and anti-glomerular basement membrane in B6.Sle1.Sle3 mice following CDDO-Me treatment (FIG. 1H-FIG. 1K). After 60 days of treatment, the serum levels of IgG anti-dsDNA, anti-single-stranded DNA (anti-ssDNA), antihistone, and antiglomerular antibodies were all significantly decreased in mice treated with CDDO-Me compared to those treated with placebo. Prior to the initiation of CDDO-Me treatment (day 0), basal levels of IgG anti-dsDNA, anti-ssDNA, anti-histone, and antiglomerular antibodies were measured and found to be comparable in the 2 groups of mice (FIG. 1H-FIG. 1K). Importantly, the most prominent benefit of this drug lies in its effective prevention of renal damage, as marked by the dramatic reduction in proteinuria, BUN, GN score, and other renal pathology measures (FIG. 1E-FIG. 1G). It is important to note that after CDDO-Me treatment, all of the IgG antibody levels listed above were reversed to normal, similar to the phenotypes previously seen in healthy B6 mice (17).

A recent clinical trial of bardoxolone methyl (another name for CDDO-Me) carried out in patients with advanced chronic kidney disease and type 2 diabetes mellitus has demonstrated its capacity in sustaining an increase in the estimated glomerular filtration rate (15). The present findings are consistent with the results of that study, and suggest that CDDO-Me might be of therapeutic benefit in chronic renal disease arising from multiple initial triggers.

### Example 5. Reduced MEK Activation in Splenic B Cells from Lupus-Prone Mice Treated with CDDO-Me

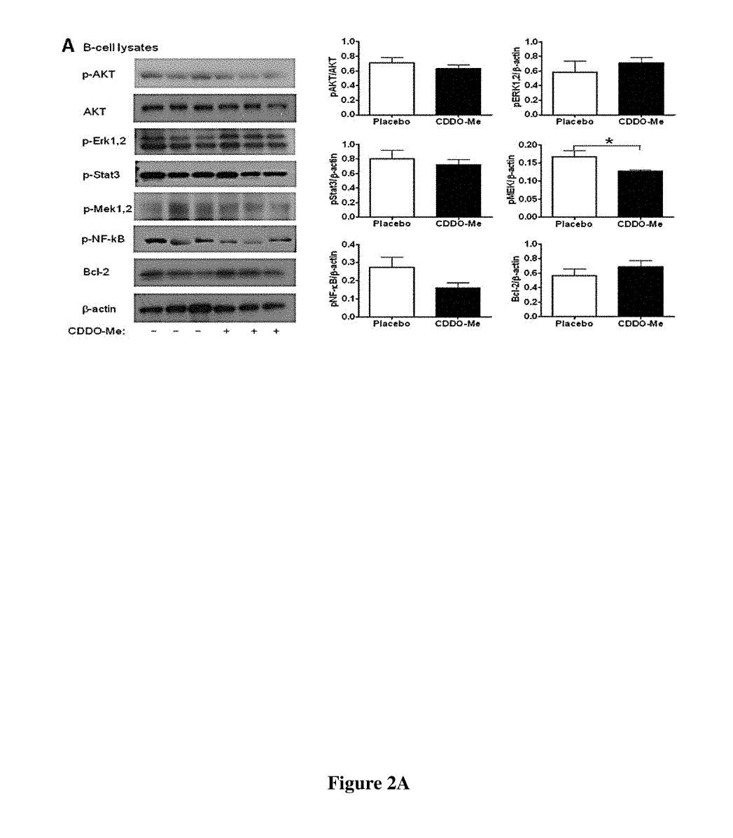

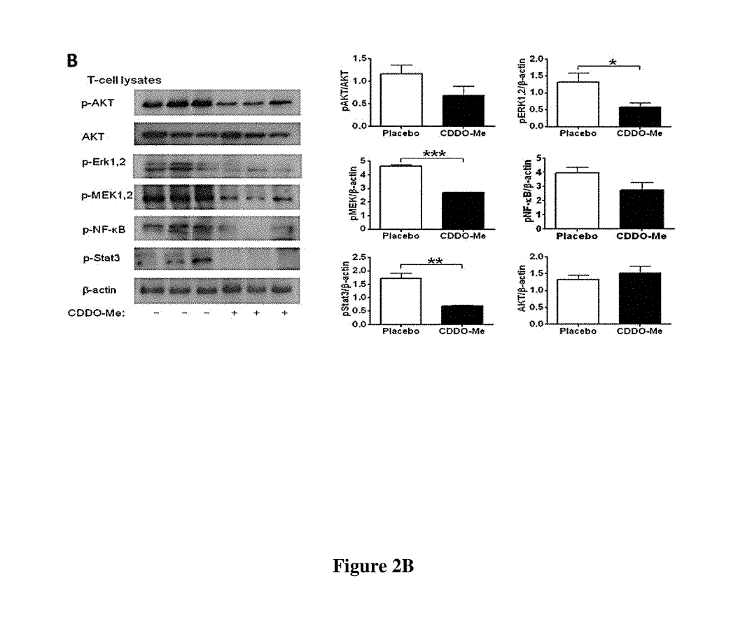

Currently, the major therapeutic use of CDDO-Me is to inactivate signaling pathways underlying cell growth and cell proliferation in cancer. To investigate the effects of CDDO-Me administration on lymphocyte signaling, splenic B220+B cells were purified with magnetic beads, and B cell lysates were analyzed by Western blot analysis for several signaling axes. The results demonstrate that MEK-1/2 activation was significantly dampened in splenic B cells from the CDDO-Me-treated mice (FIG. 2A). In addition, CDDO-Me treatment appeared to diminish the activation of NF-κB, STAT-3, and to a lesser extent Akt, although these differences were not statistically significant.

CDDO-Me treatment diminished the activation of MEK-1/2 in B cells, and of ERK, MEK, and STAT-3 in T cells. In both T cells and B cells, NF-κB showed a trend toward reduced activation following CDDO-Me treatment, but these differences did not reach statistical significance. These findings suggest that CDDO-Me can suppress cell activation and inflammatory signals mediated via multiple signaling axes not only in cancer cells, but also in immune cells and possibly in other tissues (including renal cells).

### Example 6. Reduced Activation of Selected Signaling Pathways in T Cells from Lupus-Prone Mice Treated with CDDO-Me

To determine if CDDO-Me treatment similarly impacted signaling cascades in splenic T cells, splenic CD4+T cells were isolated, and their lysates were analyzed as described above. The activation/phosphorylation of ERK-1/2, MEK-1/2, and STAT-3 were significantly ameliorated in CD4+T cells by CDDO-Me treatment (FIG. 2B). Both AKT and NF-κB showed a trend toward reduced phosphorylation, but these differences did not reach statistical significance. Taken together, the data shown in FIG. 2A and FIG. 2B provide strong evidence that multiple signaling pathways were inhibited in both splenic B cells and CD4+T cells following CDDO-Me administration.

### Example 7. CDDO-Me can Also Confer Disease Protection by Altering the Redox Pathway in Mice

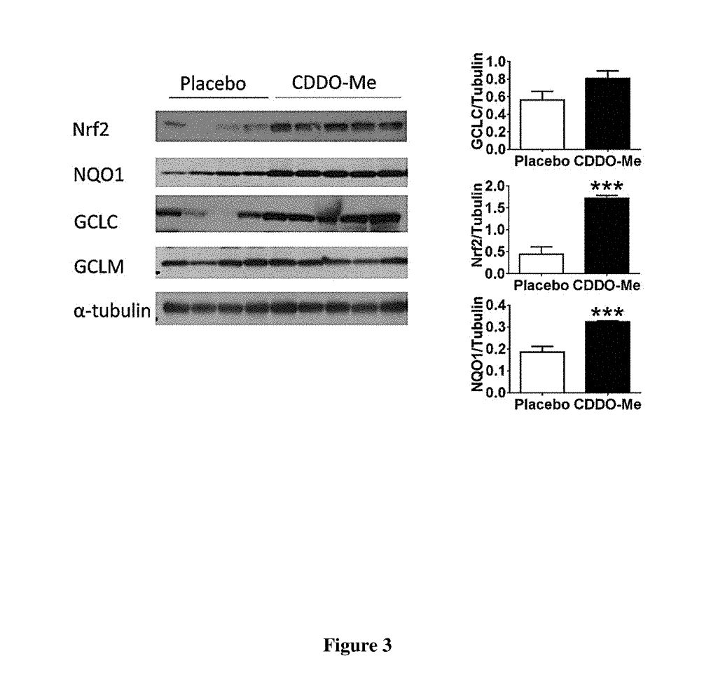

Kidney damage can be triggered via oxidative or inflammatory signals. In this context, CDDO-Me has previously been shown to protect against LPS-induced inflammatory responses via activation of the Nrf2-dependent antioxidative pathway (14). Hence, flash-frozen renal tissue from the CDDO-Me-treated mice were examined for evidence of enhanced Nrf2 activation. As shown in FIG. 3, the antioxidative modulator, Nrf2, and its target, NQO-1, were both significantly increased in the mouse kidneys after CDDO-Me treatment. The Nrf2 regulator GCLC also showed a trend toward enhanced expression after CDDO-Me treatment. These results suggest that CDDO-Me can also protect against renal damage in murine lupus nephritis by altering the intrarenal redox balance.

Besides dampening cell signaling, triterpenoids can also improve disease outcomes through other mechanisms. CDDO and its derivatives have been found to induce Nrf2 signaling, which in turn induces cytoprotective and antioxidative genes (35,36). The transcription factor Nrf2 binds and activates the antioxidant response element (37), a cis-acting sequence found in the 5′-flanking region of genes encoding many cytoprotective enzymes, including NQO-1 (38-40). It has been shown that ROS are present at higher levels during lupus nephritis (41). Therefore, antioxidant molecules such as Nrf2 and NQO-1 might be beneficial in protecting against ROS-induced kidney damage. In this study, we have shown that Nrf2 and its partner NQO-1 were significantly induced in the kidneys of B6.Sle1.Sle3 mice after CDDO-Me treatment (FIG. 3). These results suggest that renal damage and potentially other tissue damage can be ameliorated by CDDO-Me, in part via the activation of the antioxidant pathway.

The importance of Nrf2 in protecting against lupus nephritis has been reported previously. Interestingly, Nrf2-deficient female mice develop lupus-like autoimmune nephritis (42). Similar to the findings of the present study, other natural agents that are beneficial in lupus nephritis have also been associated with the elevation of renal Nrf2. Antroquinonol, a purified compound and major effective component of Antrodia camphorate, inhibited the production of ROS and nitric oxide, but increased the activation of Nrf2 within the kidneys in an accelerated mouse model of severe lupus nephritis. This was associated with significantly reduced infiltrating T cell proliferation and renal lesions (43). Epigallocatechin-3-gallate, the major bioactive polyphenol in green tea, has also been shown to increase Nrf2 and ameliorate renal disease in (NZB×NZW)F1 mice (44).

### Example 8. Validation of the Efficacy of CDDO-Me in the MRL/lpr Mouse Model of Lupus

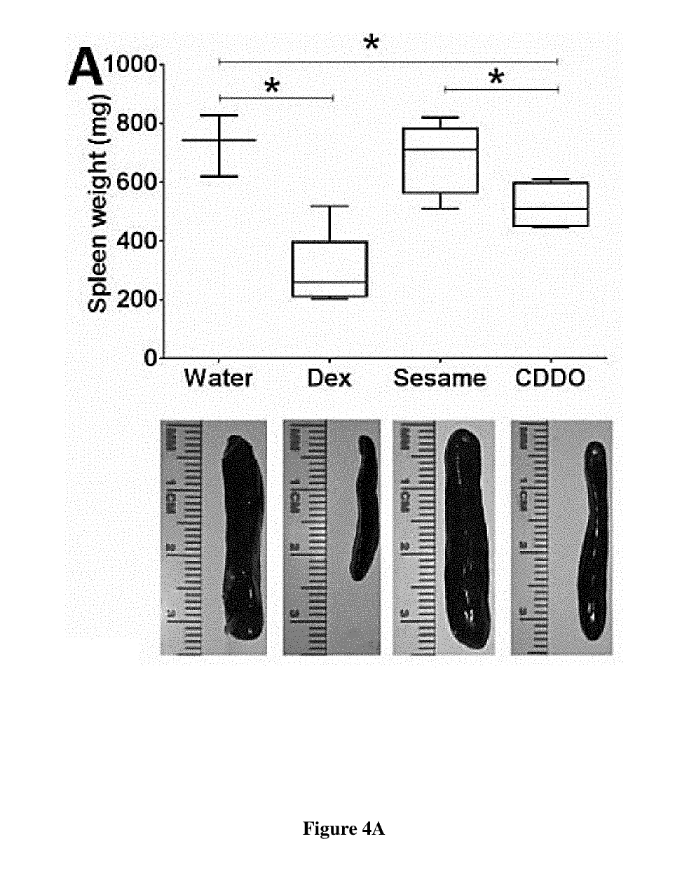

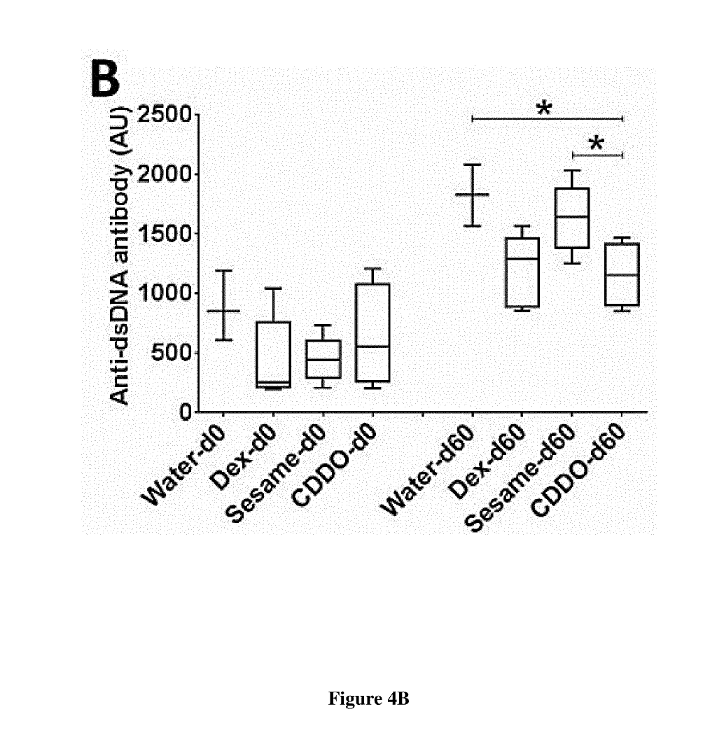

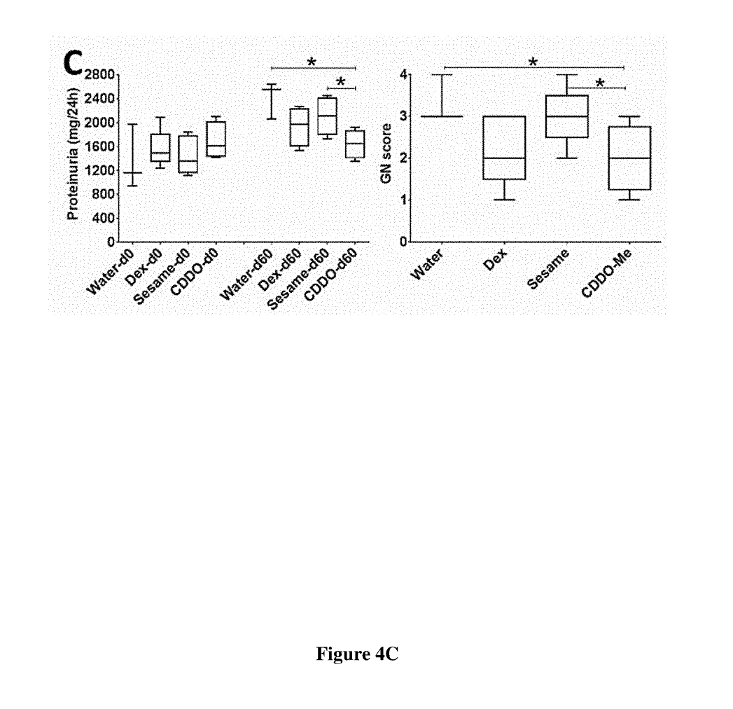

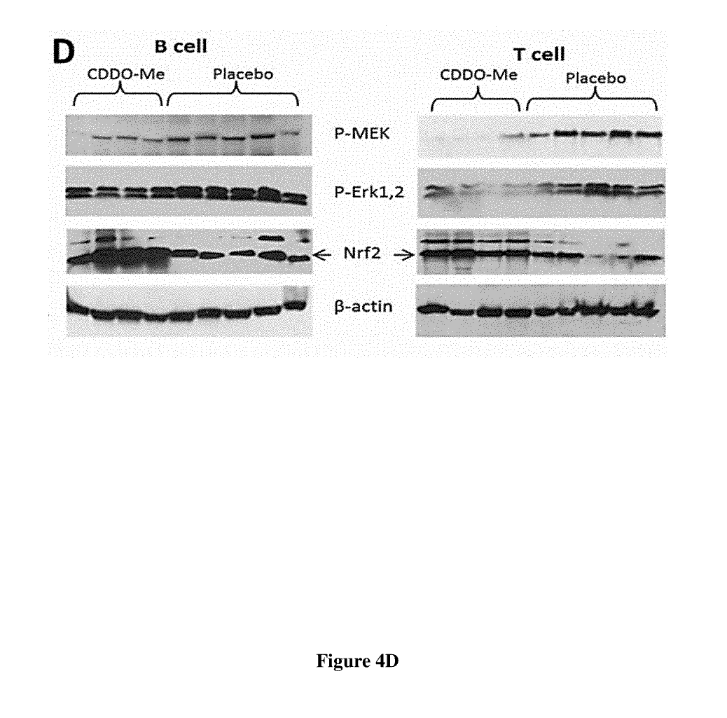

To determine if CDDO-Me attenuates disease in other murine lupus strains that are genetically different from B6.Sle1.Sle3 mice, CDDO-Me was administered to 2-month-old MRL/lpr mice, another strain that develops spontaneous lupus. In addition, to clarify if the vehicle sesame oil by itself has any modulatory effects on murine lupus, another control group that received only water was included. In this validation study, a “positive control” group for drug treatment was included, in which the lupus prone mice were treated with dexamethasone. Dexamethasone is a glucocorticoid, and some glucocorticoids are standard therapy for human lupus (19). Once again, CDDO-Me significantly reduced splenomegaly, compared to placebo (sesame oil) (FIG. 4A). As expected, a significant reduction in anti-dsDNA antibody levels was observed (FIG. 4B), proteinuria, and GN score (FIG. 4C) in the CDDO-Me-treated group compared to the control groups.

The drug control group that received dexamethasone also demonstrated disease improvement after treatment, comparable to the efficacy noted with CDDO-Me. The sesame oil vehicle by itself did not have any significant impact on the disease, since the mice treated with sesame oil exhibited phenotypes that were comparable to the mice that received water only. The signaling status in both B cells and T cells in the mouse spleen after CDDO-Me treatment was further examined. The phosphorylation of MEK and ERK-1/2 was down-regulated, whereas the antioxidative molecule Nrf2 was up-regulated in both lymphocyte compartments (FIG. 4D).

### Example 9. Mitigation of Oxidative Stress in Splenic CD4+ T Cells and CD11b+ Cells from Mice Treated with CDDO-Me

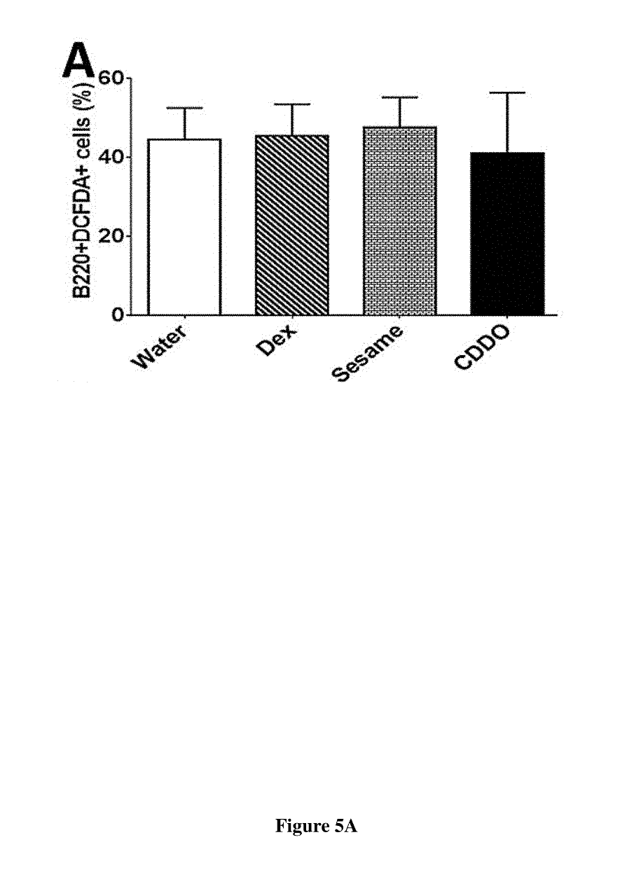

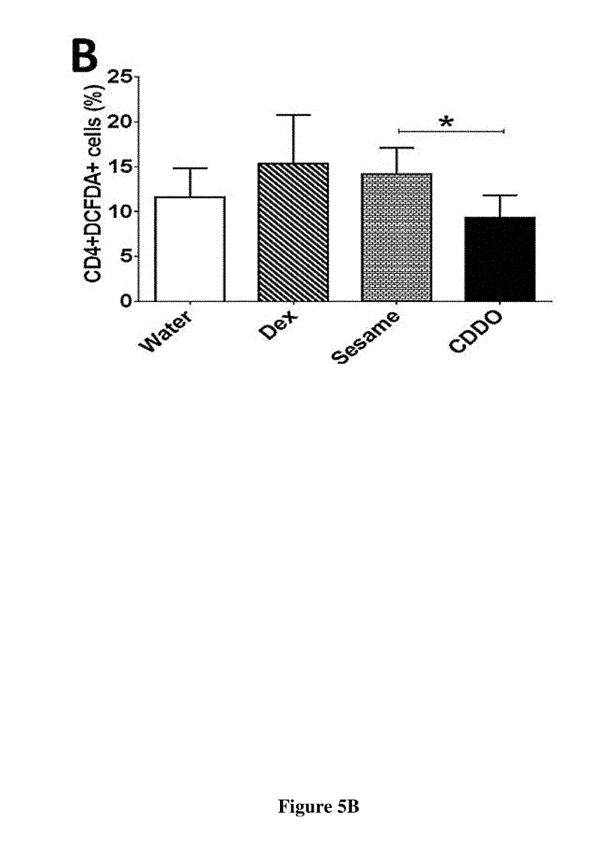

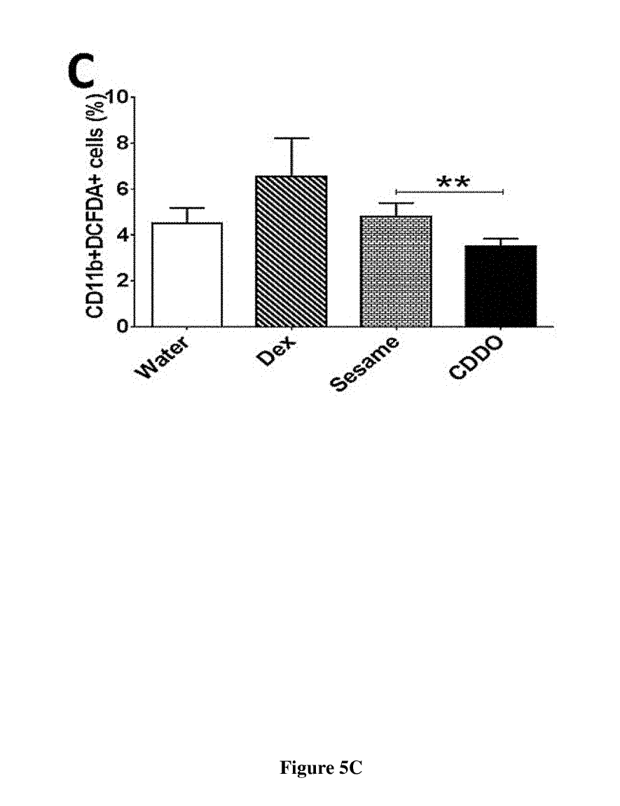

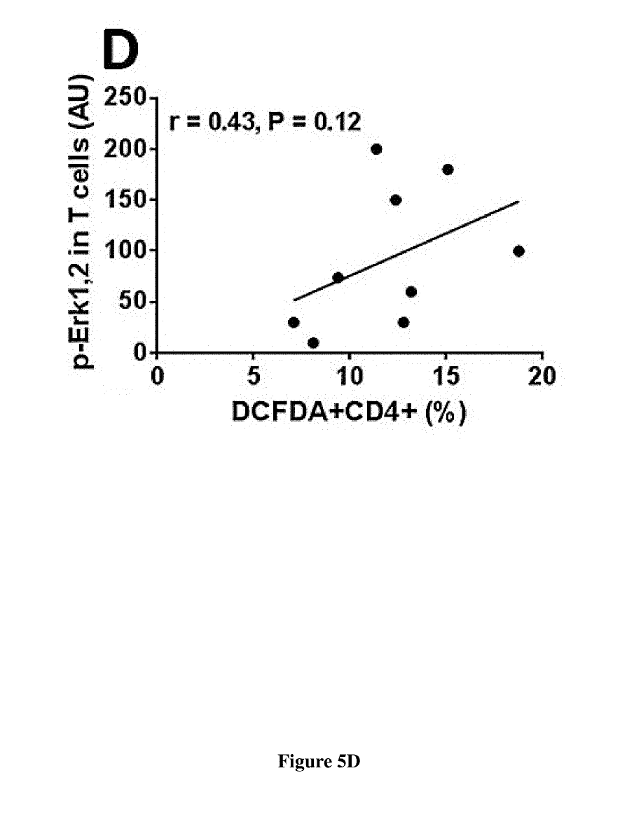

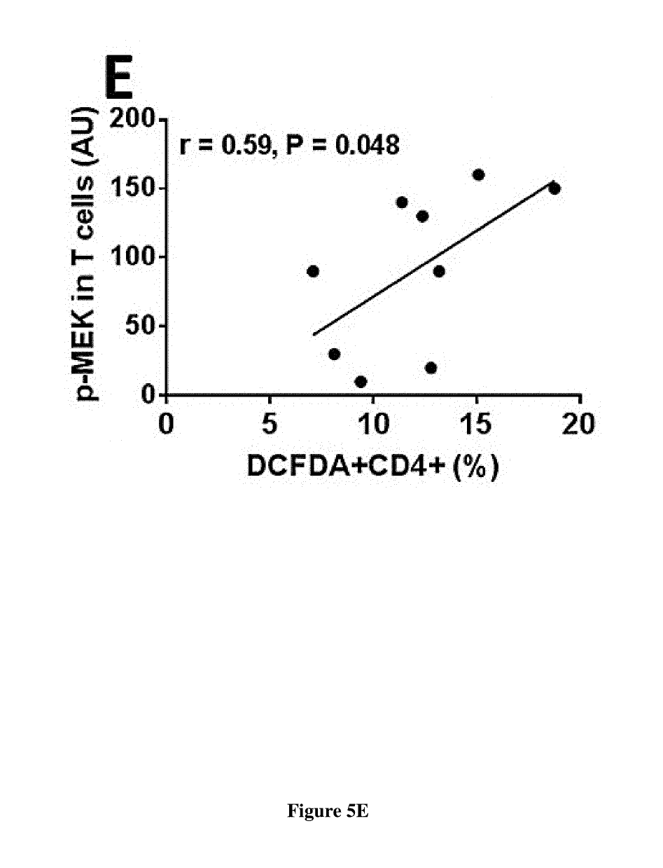

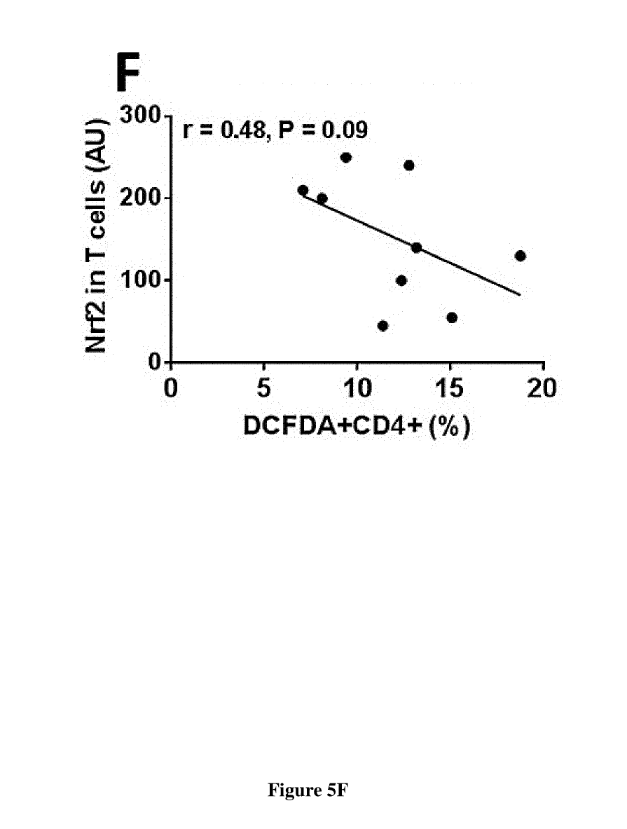

To investigate if CDDO-Me has any modulatory effect on oxidative stress in murine lupus, the level of ROS in the splenocytes using a DCFDA kit as described was examined. The ROS levels, as indicated by the percentage of DCFDA+ cells, were significantly reduced among both CD4+ T cells and CD11b+ cells in the CDDO-Me-treated group, compared to the other groups, including the dexamethasone-treated mice (FIG. 5A-FIG. 5C). In CD4+ T cells, the ROS levels positively correlated with the phosphorylation of ERK-1/2 and MEK, but negatively correlated with Nrf2 levels, suggesting that the pathways by which CDDO-Me influences lymphocyte signaling can be mechanistically linked to the pathways that generate ROS (FIG. 5D-FIG. 5F). To determine if the up-regulation or phosphorylation of the signaling molecules MEK-1 and STAT-3 can be mechanistically linked to altered ROS in murine lupus, the percentage of DCFDA+ cells among splenocytes following in vitro inhibition of MEK-1 or STAT-3 was determined; however, there did not appear to be any significant association between ROS levels and MEK-1 or STAT-3 activation.

The present study showed that CDDO-Me abrogates ROS both in vivo and in vitro (FIG. 5A-5F). The results suggest that the impact of CDDO-Me on ROS levels and lymphocyte signaling can be independent events. Thus, the inactivation of STAT-3 has been observed to suppress load-driven mitochondrial activity, leading to elevated levels of ROS in cultured primary osteoblasts (45). Conversely, ROS activates STAT-3 and induces IL-6 production in cancer cells (46). Also, treating rat sympathetic neurons with an MEK-1 inhibitor greatly decreased cellular concentrations of glutathione, a major cellular antioxidant (47). Clearly, the mechanistic links between ROS production and lymphocyte signaling warrant further study, particularly in the context of autoimmunity.

### Example 10. Attenuation of Lupus Phenotypes after Disease Onset in NZM2410 Mice Treated with CDDO-Me

Finally, to test the therapeutic efficacy of CDDO-Me after disease onset, an additional treatment experiment was performed by administering CDDO-Me to a third strain of mice with lupus (7-month-old NZM2410 mice; n=4-7 mice per group) for a period of 2 months. These mice already had proteinuria at the beginning of the study. Once again, CDDO-Me was effective in improving survival, and reducing cellularity, circulating antibodies, and proteinuria. Thus, CDDO-Me appears to be therapeutically effective even when administered after disease onset.

### Example 11. Lack of Apparent Side Effects in Mice with Lupus Treated with CDDO-Me

Since this was a relatively long-term study, and since CDDO-Me cripples multiple signaling axes, one concern can be potential side effects. After 60 days of drug administration (either in the preventive or treatment experiment), body weights of mice from both the CDDO-Me group and the placebo group were similar. Likewise, all blood cell counts, including white blood cell count, red blood cell count, hemoglobin, hematocrit, mean corpuscular volume, mean corpuscular hemoglobin, mean corpuscular hemoglobin concentration, red blood cell distribution width index, mean platelet volume, neutrophils, lymphocytes, and eosinophils remained similar between the 2 groups. Platelet counts in the 2 groups were also comparable. These results indicate that the mice experienced no apparent hematologic side effects or weight loss as a result of CDDO-Me administration. In addition, liver function was monitored by assaying aspartate aminotransferase (AST); however, there were no significant changes in the CDDO-Me-treated group (AST activity 75.7±6.5 units/liter) compared to the placebo group (74.0±5.8 units/liter).

There is a very interesting relationship between oxidative stress and one particular cell signaling pathway in lupus. The antioxidant N-acetylcysteine (NAC) has been shown to inhibit mTOR activity in vitro, and also confer therapeutic benefit in murine lupus (48,49). Consistent with the results of those earlier studies, more recent work by Lai and coworkers has also demonstrated that NAC confers therapeutic benefit in patients with SLE, once again associated with mTOR inhibition and enhanced lymphocyte apoptosis (50). It was found in the present study that Akt phosphorylation is reduced in T cells following CDDO-Me treatment. The findings suggest that one important mechanism of action of antioxidants in lupus might be reduced signaling via the Akt/mTOR axis coupled with elevated apoptosis of immune effector cells. Indeed, there is recent evidence that mTOR is a direct target of CDDO-Me (51). The relationship between oxidative stress and mTOR, and its implications for the pathogenesis and treatment of SLE, are elegantly discussed in a recent review by Perl (52).

From the foregoing description, one of ordinary skill in the art can easily ascertain the essential characteristics of this disclosure, and without departing from the spirit and scope thereof, can make various changes and modifications to adapt the disclosure to various usages and conditions. For example, we do not mean for references such as above, below, left, right, and the like to be limiting but rather as a guide for orientation of the referenced element to another element. A person of skill in the art should understand that certain of the above-described structures, functions, and operations of the above-described embodiments are not necessary to practice the present disclosure and are included in the description simply for completeness of an exemplary embodiment or embodiments. In addition, a person of skill in the art should understand that specific structures, functions, and operations set forth in the above-described referenced patents and publications can be practiced in conjunction with the present disclosure, but they are not essential to its practice.

The invention can be embodied in other specific forms without departing from its spirit or essential characteristics. A person of skill in the art should consider the described embodiments in all respects only as illustrative and not restrictive. The scope of the invention is, therefore, indicated by the appended claims rather than by the foregoing description. A person of skill in the art should embrace, within their scope, all changes to the claims which come within the meaning and range of equivalency of the claims. Further, we hereby incorporate by reference, as if presented in their entirety, all published documents, patents, and applications mentioned herein.

### REFERENCES

1. Borlado L R, Redondo C, Alvarez B, Jimenez C, Criado L M, Flores J, et al. Increased phosphoinositide 3-kinase activity induces a lymphoproliferative disorder and contributes to tumor generation in vivo. FASEB J 2000; 14:895-903.
2. Strasser A, Whittingham S, Vaux D L, Bath M L, Adams J M, Cory S, et al. Enforced BCL2 expression in B-lymphoid cells prolongs antibody responses and elicits autoimmune disease. Proc Natl Acad Sci USA 1991; 88:8661-5.
3. Di Cristofano A, Kotsi P, Peng Y F, Cordon-Cardo C, Elkon K B, Pandolfi P P. Impaired Fas response and autoimmunity in Pten^{+/-} mice. Science 1999; 285:2122-5.
4. Wu T, Qin X, Kurepa Z, Kumar K R, Liu K, Kanta H, et al. Shared signaling networks active in B cells isolated from genetically distinct mouse models of lupus. J Clin Invest 2007; 117:2186-96.
5. Sporn M B, Liby K T, Yore M M, Fu L, Lopchuk J M, Gribble G W. New synthetic triterpenoids: potent agents for prevention and treatment of tissue injury caused by inflammatory and oxidative stress. J Nat Prod 2011; 74:537-45.
6. Petronelli A, Pannitteri G, Testa U. Triterpenoids as new promising anticancer drugs. Anticancer Drugs 2009; 20:880-92.
7. Ahmad R, Raina D, Meyer C, Kufe D. Triterpenoid CDDO-methyl ester inhibits the Janus-activated kinase-1 (JAK1)-signal transducer and activator of transcription-3 (STAT3) pathway by direct inhibition of JAK1 and STAT3. Cancer Res 2008; 68:2920-6.
8. Ling X, Konopleva M, Zeng Z, Ruvolo V, Stephens L C, Schober W, et al. The novel triterpenoid C-28 methyl ester of 2-cyano-3, 12-dioxoolen-1, 9-dien-28-oic acid inhibits metastatic murine breast tumor growth through inactivation of STAT3 signaling [published erratum appears in Cancer Res 2008; 68:4958]. Cancer Res 2007; 67:4210-8.
9. Samudio I, Konopleva M, Pelicano H, Huang P, Frolova O, Bornmann W, et al. A novel mechanism of action of methyl-2-cyano-3,12 dioxoolean-1,9 diene-28-oate: direct permeabilization of the inner mitochondrial membrane to inhibit electron transport and induce apoptosis. Mol Pharmacol 2006; 69:1182-93.
10. Ahmad R, Raina D, Meyer C, Kharbanda S, Kufe D. Triterpenoid CDDO-Me blocks the NF-κB pathway by direct inhibition of IKKon Cys-179. J Biol Chem 2006; 281:35764-9.
11. Ikeda T, Sporn M, Honda T, Gribble G W, Kufe D. The novel triterpenoid CDDO and its derivatives induce apoptosis by disruption of intracellular redox balance. Cancer Res 2003; 63:5551-8.
12. Konopleva M, Tsao T, Ruvolo P, Stiouf I, Estrov Z, Leysath C E, et al. Novel triterpenoid CDDO-Me is a potent inducer of apoptosis and differentiation in acute myelogenous leukemia. Blood 2002; 99:326-35.
13. Konopleva M, Contractor R, Kurinna S M, Chen W, Andreeff M, Ruvolo P P. The novel triterpenoid CDDO-Me suppresses MAPK pathways and promotes p38 activation in acute myeloid leukemia cells. Leukemia 2005; 19:1350-4.
14. Thimmulappa R K, Fuchs R J, Malhotra D, Scollick C, Traore K, Bream J H, et al. Preclinical evaluation of targeting the Nrf2 pathway by triterpenoids (CDDO-Im and CDDO-Me) for protection from LPS-induced inflammatory response and reactive oxygen species in human peripheral blood mononuclear cells and neutrophils. Antioxid Redox Signal 2007; 9:1963-70.
15. Pergola P E, Raskin P, Toto R D, Meyer C J, Huff J W, Grossman E B, et al. Bardoxolone methyl and kidney function in CKD with type 2 diabetes. N Engl J Med 2011; 365:327-36.
16. Morel L, Rudofsky U H, Longmate J A, Schiffenbauer J, Wakeland E K. Polygenic control of susceptibility to murine systemic lupus erythematosus. Immunity 1994; 1:219-29.
17. Shi X, Xie C, Kreska D, Richardson J A, Mohan C. Genetic dissection of SLE: SLE1 and FAS impact alternate pathways leading to lymphoproliferative autoimmunity. J Exp Med 2002; 196:281-92.
18. Xie C, Zhou X J, Liu X, Mohan C. Enhanced susceptibility to end-organ disease in the lupus-facilitating NZW mouse strain. Arthritis Rheum 2003; 48:1080-92.
19. Luijten R K, Fritsch-Stork R D, Bijlsma J W, Derksen R H. The use of glucocorticoids in systemic lupus erythematosus: after 60 years still more an art than science. Autoimmun Rev 2013; 12:617-28.
20. Wong H K, Kammer G M, Dennis G, Tsokos G C. Abnormal NF-κB activity in T lymphocytes from patients with systemic lupus erythematosus is associated with decreased p65-RelA protein expression. J Immunol 1999; 163:1682-9.
21. Harada T, Kyttaris V, Li Y, Juang Y T, Wang Y, Tsokos G C. Increased expression of STAT3 in SLE T cells contributes to enhanced chemokine-mediated cell migration. Autoimmunity 2007; 40:1-8.
22. Juang Y T, Wang Y, Solomou E E, Li Y, Mawrin C, Tenbrock K, et al. Systemic lupus erythematosus serum IgG increases CREM binding to the IL-2 promoter and suppresses IL-2 production through CaMKIV. J Clin Invest 2005; 115:996-1005.
23. Krishnan S, Juang Y T, Chowdhury B, Magilavy A, Fisher C U, Nguyen H, et al. Differential expression and molecular associations of Syk in systemic lupus erythematosus T cells. J Immunol 2008; 181:8145-52.
24. Huang M T, Badmaev V, Ding Y, Liu Y, Xie J G, Ho C T. Anti-tumor and anti-carcinogenic activities of triterpenoid, betaboswellic acid. Biofactors 2000; 13:225-30.
25. Takayasu J, Tanaka R, Matsunaga S, Ueyama H, Tokuda H, Hasegawa T, et al. Anti-tumor-promoting activity of derivatives of abieslactone, a natural triterpenoid isolated from several Abies genus. Cancer Lett 1990; 53:141-4.
26. Yu L J, Ma R D, Wang Y Q, Nishino H, Takayasu J, He W Z, et al. Potent anti-tumorigenic effect of tubeimoside 1 isolated from the bulb of Bolbostemma paniculatum (Maxim) Franquet. Int J Cancer 1992; 50:635-8.
27. Nishino H, Nishino A, Takayasu J, Hasegawa T, Iwashima A, Hirabayashi K, et al. Inhibition of the tumor-promoting action of 12-O-tetradecanoylphorbol-13-acetate by some oleanane-type triterpenoid compounds. Cancer Res 1988; 48:5210-5.
28. Honda T, Rounds B V, Bore L, Favaloro F G Jr, Gribble G W, Suh N, et al. Novel synthetic oleanane triterpenoids: a series of highly active inhibitors of nitric oxide production in mouse macrophages. Bioorg Med Chem Lett 1999; 9:3429-34.
29. Kim K B, Lotan R, Yue P, Sporn M B, Suh N, Gribble G W, et al. Identification of a novel synthetic triterpenoid, methyl-2-cyano-3,12-dioxooleana-1,9-dien-28-oate, that potently induces caspase mediated apoptosis in human lung cancer cells. Mol Cancer Ther 2002; 1:177-84.
30. Shishodia S, Sethi G, Konopleva M, Andreeff M, Aggarwal B B. A synthetic triterpenoid, CDDO-Me, inhibits IκBα kinase and enhances apoptosis induced by TNF and chemotherapeutic agents through down-regulation of expression of nuclear factor κB-regulated gene products in human leukemic cells. Clin Cancer Res 2006; 12:1828-38.
31. Li M, Sun K, Redelman D, Welniak L A, Murphy W J. The triterpenoid CDDO-Me delays murine acute graft-versus-host disease with the preservation of graft-versus-tumor effects after allogeneic bone marrow transplantation. Biol Blood Marrow Transplant 2010; 16:739-50.
32. Deeb D, Gao X, Dulchaysky S A, Gautam S C. CDDO-Me inhibits proliferation, induces apoptosis, down-regulates Akt, mTOR, NF-κB and NF-κB-regulated antiapoptotic and proangiogenic proteins in TRAMP prostate cancer cells. J Exp Ther Oncol 2008; 7:31-9.
33. Deeb D, Gao X, Jiang H, Dulchaysky S A, Gautam S C. Oleanane triterpenoid CDDO-Me inhibits growth and induces apoptosis in prostate cancer cells by independently targeting pro-survival Akt and mTOR. Prostate 2009; 69:851-60.
34. Ahmad R, Liu S, Weisberg E, Nelson E, Galinsky I, Meyer C, et al. Combining the FLT3 inhibitor PKC412 and the triterpenoid CDDO-Me synergistically induces apoptosis in acute myeloid leukemia with the internal tandem duplication mutation. Mol Cancer Res 2010; 8:986-93.
35. Liby K, Hock T, Yore M M, Suh N, Place A E, Risingsong R, et al. The synthetic triterpenoids, CDDO and CDDO-imidazolide, are potent inducers of heme oxygenase-1 and Nrf2/ARE signaling. Cancer Res 2005; 65:4789-98. 36. Dinkova-Kostova A T, Liby K T, Stephenson K K, Holtzclaw W D, Gao X, Suh N, et al. Extremely potent triterpenoid inducers of the phase 2 response: correlations of protection against oxidant and inflammatory stress. Proc Natl Acad Sci USA 2005; 102:4584-9.
37. Itoh K, Chiba T, Takahashi S, Ishii T, Igarashi K, Katoh Y, et al. An Nrf2/small Maf heterodimer mediates the induction of phase II detoxifying enzyme genes through antioxidant response elements. Biochem Biophys Res Commun 1997; 236:313-22.
38. Jaiswal A K. Human NAD(P)H:quinone oxidoreductase (NQO1) gene structure and induction by dioxin. Biochemistry 1991; 30: 10647-53.
39. Nioi P, McMahon M, Itoh K, Yamamoto M, Hayes J D. Identification of a novel Nrf2-regulated antioxidant response element (ARE) in the mouse NAD(P)H:quinone oxidoreductase 1 gene: reassessment of the ARE consensus sequence. Biochem J 2003; 374:337-48.
40. Favreau L V, Pickett C B. Transcriptional regulation of the rat NAD(P)H:quinone reductase gene: identification of regulatory elements controlling basal level expression and inducible expression by planar aromatic compounds and phenolic antioxidants. J Biol Chem 1991; 266:4556-61.
41. Moroni G, Novembrino C, Quaglini S, De Giuseppe R, Gallelli B, Uva V, et al. Oxidative stress and homocysteine metabolism in patients with lupus nephritis. Lupus 2010; 19:65-72.
42. Yoh K, Itoh K, Enomoto A, Hirayama A, Yamaguchi N, Kobayashi M, et al. Nrf2-deficient female mice develop lupus-like autoimmune nephritis. Kidney Int 2001; 60:1343-53.
43. Tsai P Y, Ka S M, Chang J M, Lai J H, Dai M S, Jheng H L, et al. Antroquinonol differentially modulates T cell activity and reduces interleukin-18 production, but enhances Nrf2 activation, in murine accelerated severe lupus nephritis. Arthritis Rheum 2012; 64: 232-42.
44. Tsai P Y, Ka S M, Chang J M, Chen H C, Shui H A, Li C Y, et al. Epigallocatechin-3-gallate prevents lupus nephritis development in mice via enhancing the Nrf2 antioxidant pathway and inhibiting NLRP3 inflammasome activation. Free Radic Biol Med 2011; 51: 744-54.
45. Zhou H, Newnum A B, Martin J R, Li P, Nelson M T, Moh A, et al. Osteoblast/osteocyte-specific inactivation of Stat3 decreases load driven bone formation and accumulates reactive oxygen species. Bone 2011; 49:404-11.
46. Yoon S, Woo S U, Kang J H, Kim K, Kwon M H, Park S, et al. STATS transcriptional factor activated by reactive oxygen species induces IL6 in starvation-induced autophagy of cancer cells. Autophagy 2010; 6:1125-38.
47. Kirkland R A, Franklin J L. Prooxidant effects of NGF withdrawal and MEK inhibition in sympathetic neurons. Antioxid Redox Signal 2003; 5:635-9.
48. O'Loghlen A, Perez-Morgado M I, Salinas M, Martin M E. Nacetyl-cysteine abolishes hydrogen peroxide-induced modification of eukaryotic initiation factor 4F activity via distinct signaling pathways. Cell Signal 2006; 18:21-31.
49. Suwannaroj S, Lagoo A, Keisler D, McMurray R W. Antioxidants suppress mortality in the female NZB NZW F1 mouse model of systemic lupus erythematosus (SLE). Lupus 2001; 10:258-65.
50. Lai Z W, Hanczko R, Bonilla E, Caza T N, Clair B, Bartos A, et al. N-acetylcysteine reduces disease activity by blocking mammalian target of rapamycin in T cells from systemic lupus erythematosus patients: a randomized, double-blind, placebo-controlled trial. Arthritis Rheum 2012; 64:2937-46.
51. Yore M M, Kettenbach A N, Sporn M B, Gerber S A, Liby K T. Proteomic analysis shows synthetic oleanane triterpenoid binds to mTOR. PloS One 2011; 6:e22862.
52. Perl A. Oxidative stress in the pathology and treatment of systemic lupus erythematosus. Nat Rev Rheumatol 2013; 9:674-86.

What is claimed is:

1. A method of treatment of a subject with an autoimmune disease comprising administering CDDO-Me to a subject, wherein the treatment results in suppression of activation of lymphocytes.

2. The method of claim 1, wherein the lymphocytes are CD4+ T cells.

3. The method of claim 1, wherein the lymphocytes are B cells.

4. The method of claim 1, wherein administering CDDO-Me blocks signaling pathways in lymphocytes.

5. The method of claim 1, wherein administering CDDO-Me inhibits at least one selected from the group consisting of AKT, MEK-1/2, NF-κB and a combination thereof in lymphocytes.

6. The method of claim 1, wherein administering CDDO-Me induces apoptosis of the lymphocytes.

7. The method of claim 1, wherein administering CDDO-Me reduces autoantibody production.

8. The method of claim 1, wherein administering CDDO-Me reverses lupus nephritis by activation of a NRF2-mediated antioxidative pathway.

9. The method of claim 1, wherein the autoimmune disease is lupus.

10. The method of claim 1, wherein the autoimmune disease is graft versus host disease.

11. The method of claim 1, wherein the autoimmune disease is lupus nephritis

12. A method of prevention of an autoimmune disease in a subject using CDDO-Me, comprising administering CDDO-Me to a subject, wherein the CDDO-Me suppresses activation of lymphocytes.

13. The method of claim 12, wherein the lymphocytes are CD4+ T cells.

14. The method of claim 12, wherein the lymphocytes are B cells.

15. The method of claim 12, wherein administering CDDO-Me inhibits in lymphocytes at least one selected from the group consisting of AKT, MEK-1/2, NF-κB and a combination thereof.

16. The method of claim 12, wherein the CDDO-Me induces apoptosis of the lymphocytes.

17. The method of claim 12, wherein the CDDO-Me reduces autoantibody production.

18. The method of claim 12, wherein the autoimmune disease is lupus.

19. The method of claim 12, wherein the autoimmune disease is graft versus host.

20. The method of claim 12, wherein the autoimmune disease is lupus nephritis.
